(19) United States
(12) Patent Application Publication
(10) Pub. No.: US 2006/0134048 A1
(43) Pub. Date: Jun. 22, 2006

(54) Reduction of hair growth

(76) Inventors: Douglas Shander, Acton, MA (US); James P. Henry, Mendon, MA (US); Hsing-Mei Wu, Newton, MA (US); Natalia Botchkareva, Sharon, MA (US); Gurpreet S. Ahluwalia, Wellesley, MA (US); Mark W. Trumbore, Westford, MA (US)

Correspondence Address: FISH & RICHARDSON P.C., P.O. BOX 1022, MINNEAPOLIS, MN, 55440-1022, US

(21) Appl. No.: 11/313,986

(22) Filed: Dec. 21, 2005

Related U.S. Application Data
(60) Provisional application No. 60/639,069, filed on Dec. 22, 2004.

(51) Int. Cl. A61K 48/00; A61K 8/73

(52) U.S. Cl. 424/70.13; 514/44

(57) ABSTRACT

A method of reducing hair growth includes topical application of a heat shock protein inhibitor and/or a compound that promotes apoptosis in conjunction with heat.

### CLAIM OF PRIORITY

This application claims priority under 35 U.S.C. § 119(e) to U.S. patent application Ser. No. 60/639,069, filed on Dec. 22, 2004, the entire contents of which are hereby incorporated by reference.

### BACKGROUND

The invention relates to reducing hair growth in mammals, particularly for cosmetic purposes.

A main function of mammalian hair is to provide environmental protection. However, that function has largely been lost in humans, in whom hair is kept or removed from various parts of the body essentially for cosmetic reasons. For example, it is generally preferred to have hair on the scalp but not on the face.

Various procedures have been employed to remove unwanted hair, including shaving, electrolysis, depilatory creams or lotions, waxing, plucking, and therapeutic antiandrogens. These conventional procedures generally have drawbacks associated with them. Shaving, for instance, can cause nicks and cuts, and can leave a perception of an increase in the rate of hair regrowth. Shaving also can leave an undesirable stubble. Electrolysis, on the other hand, can keep a treated area free of hair for prolonged periods of time, but can be expensive, painful, and sometimes leaves scarring. Depilatory creams, though very effective, typically are not recommended for frequent use due to their high irritancy potential. Waxing and plucking can cause pain, discomfort, and poor removal of short hair. Finally, antiandrogens—which have been used to treat female hirsutism—can have unwanted side effects.

It has previously been disclosed that the rate and character of hair growth can be altered by applying to the skin inhibitors of certain enzymes. These inhibitors include inhibitors of 5-alpha reductase, ornithine decarboxylase, S-adenosylmethionine decarboxylase, gamma-glutamyl transpeptidase, and transglutaminase. See, for example, Breuer et al., U.S. Pat. No. 4,885,289; Shander, U.S. Pat. No. 4,720,489; Ahluwalia, U.S. Pat. No. 5,095,007; Ahluwalia et al., U.S. Pat. No. 5,096,911; and Shander et al., U.S. Pat. No. 5,132,293.

Heat shock proteins (HSPs) are a known superfamily of evolutionary conserved proteins, which consist of sub-families with different molecular weight. Examples of HSPs include HSP-27, HSP-70, and HSP-90. HSPs perform multiple intracellular functions. They are also called “stress proteins”, because their synthesis is stimulated by variety of stresses, including cytotoxic drugs, heat, and irradiation. HSPs may play a role in maintenance of cellular homeostasis under physiological conditions as well. Synthesis of HSPs occurs as a result of transcriptional activation of responsive elements by heat shock specific transcription factors, inhibition of which leads to decrease in the level of HSPs. HSPs act as chaperone molecules that bind to client proteins to facilitate their proper folding, assist protein transport and sorting between intracellular compartments, and control their switching between active/native conformation. Among the substrates of HSPs are a number of tyrosine, serine/threonine, and cyclin dependent kinases. In addition, HSP-90 is involved in modulating signaling through hormone receptors. Interactions of HSPs with their dependent proteins are required for regulation of cell proliferation and differentiation.

In addition to cell cycle regulation, HSPs may protect cells against programmed cell death, referred as apoptosis, induced by wide variety of stimuli. HSPs possess substantial anti-apoptotic properties. They may control programmed cell death at different intracellular levels. Overexpression of HSPs may protect cells against apoptosis induced by Fas, TNF, ceramide, and cytotoxic drugs. It was shown that HSPs are involved in mitochondria dependent apoptotic pathways, preventing activation of caspases. HSP70 and HSP-90 interact with mutant p53, causing decrease in wild type p53, which is important regulator of cell cycle arrest/apoptosis.

Apoptosis is programmed cell death. The apoptosis process creates an appropriate balance between cellular proliferation and death. In the case of hair follicle cells, apoptosis appears to be involved in the regulation of hair growth and hair cycle.

### SUMMARY

In one aspect, the invention provides a method (typically a cosmetic method) of reducing unwanted mammalian (preferably human) hair growth. The method includes applying a composition including a heat shock protein (HSP) inhibitor or a compound that promotes apoptosis to the area of skin, and heating the area of skin within 14 days of applying the composition. The appropriate time period depends on the specific chemical agent, and could be as short as a day or less, and treatment can even be simultaneous. The heating may be performed using, for example, a laser or flashlamp. Preferably the composition is applied multiple times and heating is performed within seven days, and more preferably within three days or one day, or simultaneously with, one of the applications. The unwanted hair growth may be undesirable from a cosmetic standpoint or may result, for example, from a disease or an abnormal condition (for example, hirsutism).

HSP inhibitors include compounds that inhibit the activity of one or more hair follicle HSPs by strongly interacting with the HSP(s); compounds that reduce the levels and/or expression of one or more HSPs in hair follicles; and/or compounds that reduce the expression of one or more HSP mRNA's in hair follicles. “Strongly interacts” means the compound binds or preferentially binds to the HSP(s).

Typically, in practicing the aforementioned methods the composition will also include a dermatologically or cosmetically acceptable vehicle. Accordingly, the present invention also relates to topical compositions comprising a dermatologically or cosmetically acceptable vehicle and an HSP inhibitor or a compound that promotes apoptosis.

In addition, the present invention relates to the use of an HSP inhibitor or a compound that promotes apoptosis for the manufacture of a therapeutic topical composition. The composition optionally may include both an HSP inhibitor and a compound that promotes apoptosis.

In another aspect, the invention provides a method (typically a cosmetic method) of reducing unwanted mammalian (preferably human) hair growth. The method includes applying a composition including an HSP inhibitor or a compound that promotes apoptosis to the area of skin, and treating the area of skin with a laser, flashlamp, or an IPL device within 14 days of applying the composition. Embodiments of this aspect of the invention may further include one or more of the features discussed above.

In another aspect, the invention provides a method (typically a cosmetic method) of reducing unwanted mammalian (preferably human) hair growth. The method includes applying a composition including a compound that promotes apoptosis. The compound can be selected, for example, from one of the types of compounds discussed below.

In another aspect, the invention provides a method (typically a cosmetic method) of reducing unwanted mammalian (preferably human) hair growth. The method includes applying a composition including a compound that reduces hair growth. The compound may be, for example, an inhibitor of an enzyme, a sulfhydryl active compound (for example, cysteamine, D-penicillamine, N-acetyl cysteine, and thiosalicylic acid), a catechin compound (for example, epigallocatechingallate or EGCG) or an angiogenesis inhibitor (for example, bathocuproine, mycophenolic acid, and tamoxifen). The enzyme (or its inhibitor) may be, for example, ornithine decarboxylase (for example, alpha-difluoromethylornithine or DFMO), lipoxygenase (quercetin, propyl gallate, NDGA and caffeic acid), matrix metalloproteinase (for example, minocycline, tetracycline, and doxacycline), cyclooxygenase (for example, ibuprofin, naproxen, ketoprofen, indomethacin, and sulindac), HMG Co-A reductase (lovastatin, simivistatin, and mevastatin), gamma-glutamyl transpeptidase (for example, anthglutin, and acivicin), and transglutaminase (for example, 5-(N-benzyloxycarbonyl-L-phenylalaninamidomethly)-3-bromo-4,5-dihydroisoxazole). The compound may also be an antagonist of Vanilloid receptor-1 (VR-1), for example, Capsazepine. The method further includes treating the area of skin with a laser, a flashlamp, or an IPL device within 14 days of applying the composition. The area can be treated, for example, within seven days, two days, or one day subsequent to applying the composition. The composition can be applied, for example, at least every other day for at least two weeks after treatment with the laser, flashlamp, or ISP device. Embodiments of this aspect of the invention may include one or more of the features discussed above.

In another aspect, the invention provides a method (typically a cosmetic method) of reducing unwanted mammalian (preferably human) hair growth. The method includes treating an area of skin with a laser, a flashlamp, or an ISP device, and after step (b) applying to the area of skin, at least every other day for at least two weeks, a composition that reduces hair growth in an amount effective to reduce hair growth. The energy provided by the laser may be, for example, less than 10 J/cm². The compound can be, for example, any of the compounds mentioned above.

The above methods preferably include an initial step of selecting the area of skin from which the reduced hair growth is desired.

Specific compounds mentioned herein include both the compound itself and pharmaceutically acceptable salts thereof.

Other features and advantages of the invention will be apparent from the detailed description, and from the claims.

### DETAILED DESCRIPTION

An example of a composition includes an HSP inhibitor or a compound that promotes apoptosis in a cosmetically and/or dermatologically acceptable vehicle. The composition optionally may include both an HSP inhibitor and a compound that promotes apoptosis. The composition may be a solid, semi-solid, or liquid. The composition may be, for example, a cosmetic and dermatologic product in the form of an, for example, ointment, lotion, foam, cream, gel, or solution. The composition may also be in the form of a shaving preparation or an aftershave. The vehicle itself can be inert or it can possess cosmetic, physiological and/or pharmaceutical benefits of its own.

Examples of known HSP inhibitors are provided in Table 1.

TABLE 1

| Name of Inhibitor | Chemical Name | Function | References |
|---|---|---|---|
| Geldanamycin | 2-Azabicyclo[16.3.1]docosa-4,6,10,18,21- | Inhibitor of | Stebbins, C. et al., Cell (1997), |
| | pentaene-3,20,22-trione, 9,13-dihydroxy-8,14,19- | HSP-90 | 89, 239-250. |
| | trimethoxy-4,10,12,16-tetramethyl-,9-carbamate | activity | Roe, S. Mark et al., Journal of |
| | (8CI); | | Medicinal Chemistry (1999), |
| | 2-Azabicyclo[16.3.1]docosane, geldanamycin | | 42, 260-266. |
| | deriv.; | | |
| | 2-Azabicyclo[16.3.1]docosa-4,6,10,18,21- | | |
| | pentaene-3,20,22-trione,9-[(aminocarbonyl)oxy]- | | |
| | 13-hydroxy-8,14,19-trimethoxy-4,10,12,16- | | |
| | tetramethyl-,[8S- | | |
| | (4E,6Z,8R*,9R*,10E,12R*,13S*,14R*,16S*)]-; | | |
| | NSC 122750; NSC 212518; | | |
| | [8S-(4E,6Z,8R*,9R*,10E,12R*, | | |
| | 13S*,14R*,16S*)]-9-[(Aminocarbonyl)oxy]-13- | | |
| | hydroxy-8,14,19-trimethoxy-4,10,12,16- | | |
| | tetramethyl-2-azabicyclo[16.3.1]docosa- | | |
| | 4,6,10,18,21-pentaene-3,20,22-trione | | |
| 17-Allylamino,17- | 2-Azabicyclo[16.3.1] docosane, geldanamycin | Inhibitor of | Hostein et al., Cancer |
| demethoxygeldanamycin | deriv.; | HSP-90 | Research (2001), 61, 4003-4009. |
| | 17-Amino-17-demethoxygeldanamycin | activity | |
| KF25706, KF58333, | Oxime derivatives of radicicol; radicicol 6-oxime | Inhibitor of | Soga S et al., Cancer Res. |
| KF58332 | | HSP-90 | 1999 Jun 15; 59(12): 2931-8. |
| | | activity | Shiotsu et al., Blood. 2000 Sep |
| | | | 15; 96(6): 2284-91. |
| | | | Soga et al., 2003 |
| | | | Oct; 3(5): 359-69. Review. |
| O-carbamoylmethyloxime | O-carbamoylmethyloxime derivatives of radicicol | Inhibitor of | Ikuina Y, et al., J Med Chem. |
| | | HSP-90 | 2003 Jun 5; 46(12): 2534-41. |
| | | activity | |
| Benzo-1,3-dioxole | Benzo-1,3-dioxole | Inhibitor of | U.S. Pat. No. 6,613,780. |
| | | Heat Shock | |
| | | Factor | |
| | | activity, | |
| | | which leads | |
| | | to inhibition | |
| | | of HSP | |
| | | synthesis | |
| KNK 437 | 1-Pyrrolidinecarboxaldehyde,3-(1,3- | Inhibitors | Koishi et al., Clinical Cancer |
| | benzodioxol-5-ylmethylene)-2-oxo-(9CI) | of HSP | Research (2001), 7, 215-219. |
| KNK423 | N-Formyl-3,4-methylenedioxy-benzylidine-γ- | synthesis | Yokota et al., Cancer Research |
| | butyrolactam | | (2000), 60(11), 2942-2948. |
| | 3,4-Methylenedioxy-benzylidine-γ-butyrolactam | | Yokota et al., PCT WO/98- |
| | | | JP2829 19980625. |
| ADD70-1 Protein | Synthetic gene AIF fragment (9CI) | Inhibitor of | Cancer Res. 2003; 63: 8233-40. |
| | | HSP-70 | |
| | | activity | |
| PU24FCI | | Inhibitor of | Chem Biol. 2004; 11: 787-97. |
| | | HSP-90 | |
| | | activity | |

Compounds that promote apoptosis and the cellular targets via which the apoptosis induction is achieved are shown in Table 2. Table 2.1 and 2.2 lists inhibitors and activators of apoptotic targets, respectively.

TABLE 2.1

| Targets | Chemicals | References |
|---|---|---|
| Bcl-2 | Oblimersen sodium (genasense, g3139), | Clin Cancer Res. 2004; 10: 5048-57. |
| (B-cell lymphoma/leukemia-2) | an antisense oligonucleotide to the bcl-2 | |
| | mRNA | |
| | HA14-1 analogues | Mol Cancer Ther. 2002; 1: 961-7. |
| | CPM-1285 analogues | Cancer Res. 2000; 60: 1498-502. |
| | BH3I-1\|BH3I-2 | Cancer Res. 2004; 64: 3607-16. |
| | Antimycin A3 | Nat Cell Biol. 2001; 3: 183-91. |
| | Terphenyl derivative | J Am Chem Soc. 2002; 124: 11838-9. |
| | Apogossypol | Chem Biol. 2004; 1: 389-95. |
| | Theaflavin (tea polyphenol) | Cancer Res. 2003; 63: 8118-21. |
| | SAHBs (the stapled peptides, called | Science. 2004; 305: 1466-70. |
| | “stabilized alpha-helix of BCL-2 | |
| | domains”) | |
| IAP | Benzenesulphonamide derivatives | Chem Biol. 2003 Aug; 10(8): 759-67. |
| (inhibitor of apoptosis proteins) | Capped tripeptides containing unnatural | J Med Chem. 2004 Aug 26; 47(18): 4417-26. |
| | amino acids | |
| | Embeline | J Med Chem. 2004 May 6; 47(10): 2430-40. |
| | Di/triphenylureas (1396-11,12,34) | Cancer Cell. 2004 Jan; 5(1): 25-35. |
| Bcl2 —NUR77/TR3 | 3Cl-AHPC/MM11453(6-[3-(1- | Cancer Res. 2001 Jun 15; 61(12): 4723-30 |
| (orphan nuclear receptor)interactions | adamantyl)-4-hydroxyphenyl]-2- | |
| | naphthalenecarboxylic acid) | |
| MDM2 | Nutlins | Cell Cycle. 2004; 3: 419-21 |
| | Chlorofusin (an inhibitor of p53-MDM2 | Chem Commun (Camb). 2003; 7: 316-7 |
| | interactions) | |
| | 2-phenoxybenzoyl-tryptophan | Anal Biochem. 2004 Aug 1; 331(1): 138-46 |
| | derivatives | |
| | Sulphonamide compound 1 | J Med Chem. 2004 Aug 12; 47(17): 4163-5 |
| FLIP | CDDO (triterpenoid 2-cyano-3,12- | Blood. 2002; 100: 2965-72 |
| FLICE (FADD-homologous ICE/CED- | dioxoolean-1,9-dien-28-oic acid) | |
| 3-like protease)-inhibitory protein | | |
| AKT/PKB | API-2 (Akt/protein kinase B signaling | Cancer Res. 2004; 64: 4394-9 |
| (protein kinase B) | inhibitor-2) | |
| | DPIs: DPI 1-[(R)-2,3- | Mol Cancer Ther. 2003; 2(4): 389-99 |
| | bis(hexadecanoyloxy)propyl hydrogen | |
| | phosphate], lipid derivative DPI 1-[(R)- | |
| | 2-methoxy-3-octadecyloxypropyl | |
| | hydrogen phosphate] (DPIEL), and | |
| | carbonate derivative DPI 1-[(R)-2- | |
| | methoxy-3-octadecyloxypropyl | |
| | carbonate]. | |
| 26S proteasome | 1L-6-hydroxymethyl-chiro-inositol 2(R)- | Leukemia. 2003; 17(9): 1794-805 |
| | 2-O-methyl-3-O-octadecylcarbonate | |
| | MG-115: L-leucinamide, N- | Prostate Cancer Prostatic Dis. |
| | [(phenylmethoxy)carbonyl]-L-leucyl-N- | 2004; 7(2): 138-43 |
| | [(1S)-1-formylbutyl]-(9CI), or L- | |
| | leucinamide, N- | |
| | [(phenylmethoxy)carbonyl]-L-leucyl-N- | |
| | (1-formylbutyl)-, (S)- | |
| | MG-132; L-Leucinamide, N- | Oncogene. 2004 Apr 1; 23(14): 2554-8 |
| | [(phenylmethoxy)carbonyl]-L-leucyl-N- | |
| | [(1S)-1-formyl-3-methylbutyl]-(9CI)L- | |
| | Leucinamide, N- | |
| | [(phenylmethoxy)carbonyl]-L-leucyl-N- | |
| | (1-formyl-3-methylbutyl)-(S)- | |
| | Bortezomib (Velcade) | Clin Lymphoma. 2002; 3(1): 49-55 |
| | Lactacystin | Eur J Haematol. 2000 Oct; 65(4): 221-36 |
| | Epoxomicin | Chem Biol. 2001; 8(9): 913-29 |
| IkappaB kinase (IKK) | Pyridyl cyanoguanidine derivative (CHS | Int J Cancer. 2004 Aug 20; 111(2): 198-205 |
| | 828) | |
| | Benzo-imidazole carboxamide derivative | Int J Obes Relat Metab Disord. 2004 |
| | | Aug; 28(8): 985-92 |
| | Indole carboxamide derivativeS1627 | J Neurosci. 2004 Feb 18; 24(7): 1637-45. |
| | Ureido-thiophene carboxamide | Bioorg Med Chem Lett. 2004 Jun |
| | derivative | 7; 14(11): 2817-22. |
| | 2-amino-6-[2-(cyclopropylmethoxy)-6- | Bioorg Med Chem Lett. 2004 Aug |
| | hydroxy-phenyl]-4-piperidin-4-yl | 2; 14(15): 4019-22. |
| | nicotinonitrile | |
| | Beta-carboline | Bioorg Med Chem Lett. 2003 Jul |
| | | 21; 13(14): 2419-22. |
| | BMS-345541 (4(2′-aminoethyl)amino- | J Biol Chem. 2003 Jan 17; 278(3): 1450-6. |
| | 1,8-dimethylimidazo(1,2-a)quinoxaline) | Epub 2002 Oct 25. |
| | Pyridooxazinone derivative | Bioorg Med Chem Lett. 2003 Mar |
| | | 10; 13(5): 913-8. |
| | SC-514 [2,3′-Bithiophene]-5- | J Biol Chem. 2003 Aug |
| | carboxamide, 4-amino-(9CI) | 29; 278(35): 32861-71. Epub 2003 Jun |
| | [2,3′]bithiophenyl-5-carboxamide | 17. |
| | Ethyl 2-[(3-methyl-2,5-dioxo(3- | Bioorg Med Chem Lett. 2002 Sep |
| | pyrrolinyl))-N-methylamino]-4- | 16; 12(18): 2573-7. |
| | (trifluoromethyl)-pyrimidine-5- | |
| | carboxylate (SPC-839) | |

TABLE 2.2

| | | |
|---|---|---|
| BAX | 3,6-Dibromocarbazole piperazine | J Med Chem. 2003; 46: 4365-8 |
| | derivatives of 2-propanol | |
| p53 | CP-31398; 1,3-Propanediamine, N′-[2- | J Negat Results Biomed. 2004; 3:5 |
| | [2-(4-methoxyphenyl)ethenyl]-4- | |
| | quinazolinyl]-N,N-dimethyl-(9CI) | |
| pan-TRAILR | Apo2L/TRIAL | J Clin Invest. 1999 Jul; 104(2): 155-62 |
| (tumor necrosis factor-related apoptosis- | | |
| inducing ligand receptor) | | |
| APAF1/caspases | Alpha-(trichloromethyl)-4- | Science. 2003; 299: 223-6. |
| (apoptotic protease activating factor 1) | pyridineethanol (PETCM) | |

Additional examples of apoptosis inducing agents include methylxanthines such as caffeine, theophylline, and pentoxifylline.

The composition may include more than one HSP inhibitor and/or compound that promote apoptosis. The composition also may include one or more other types of hair growth reducing agents, such as those described in U.S. Pat. No. 4,720,489; U.S. Pat. No. 4,885,289; U.S. Pat. No. 5,095,007; U.S. Pat. 5,096,911; U.S. Pat. No. 5,132,293; U.S. Pat. No. 5,143,925; U.S. Pat. No. 5,328,686; U.S. Pat. No. 5,364,885; U.S. Pat. No. 5,411,991; U.S. Pat. No. 5,440,090; U.S. Pat. No. 5,468,476; U.S. Pat. No. 5,475,763;; U.S. Pat. No. 5,554,608; U.S. Pat. No. 5,648,394; U.S. Pat. 5,652,273; U.S. Pat. No. 5,674,477; U.S. Pat. No. 5,728,736; U.S. Pat. No. 5,776,442; U.S. Pat. No. 5,824,665; U.S. Pat. No. 5,840,752; U.S. Pat. No. 5,908,867; U.S. Pat. No. 5,939,458; U.S. Pat. No. 5,958,946; U.S. Pat. No. 5,962,466; U.S. Pat. No. 6,020,006; U.S. Pat. No. 6,037,326; U.S. Pat. No. 6,060,471; U.S. Pat. No. 6,093,748; U.S. Pat. No. 6,121,269; U.S. Pat. No. 6,218,435; U.S. Pat. No. 6,235,737; U.S. Pat. No. 6,239,170; U.S. Pat. No. 6,248,751;U.S. Pat. No. 6,299,865; U.S. Pat. No. 6,414,017; U.S. Pat. No. 6,743,419; and U.S. Pat. No. 6,743,822, all of which are incorporated herein by reference.

The concentration of the HSP inhibitor or compound that promotes apoptosis in the composition may be varied over a wide range up to a saturated solution, preferably from 0.1% to 30% by weight or even more; the reduction of hair growth increases as the amount applied increases per unit area of skin. The maximum amount effectively applied is limited only by the rate of penetration of the skin. The effective amounts may range, for example, from 10 to 3000 micrograms or more per square centimeter of skin.

The vehicle can be inert or can possess cosmetic, physiological and/or pharmaceutical benefits of its own. Vehicles can be formulated with liquid or solid emollients, solvents, thickeners, humectants and/or powders. Emollients include stearyl alcohol, mink oil, cetyl alcohol, oleyl alcohol, isopropyl laurate, polyethylene glycol, petroleum jelly, palmitic acid, oleic acid, and myristyl myristate. Solvents include ethyl alcohol, isopropanol, acetone, diethylene glycol, ethylene glycol, dimethyl sulfoxide, and dimethyl formamide.

The composition optionally can include components that enhance the penetration of the HSP inhibitor and/or compound that promotes apoptosis into the skin and/or to the site of action. Examples of penetration enhancers include urea, polyoxyethylene ethers (e.g., Brij-30 and Laureth-4), 3-hydroxy-3,7,11-trimethyl-1,6,10-dodecatriene, terpenes, cis-fatty acids (e.g., oleic acid, palmitoleic acid), acetone, laurocapram, dimethylsulfoxide, 2-pyrrolidone, oleyl alcohol, glyceryl-3-stearate, propan-2-ol, myristic acid isopropyl ester, cholesterol, and propylene glycol. A penetration enhancer can be added, for example, at concentrations of 0.1% to 20% or 0.5% to 5% by weight.

The composition also can be formulated to provide a reservoir within or on the surface of the skin to provide for a continual slow release of the HSP inhibitor and/or compound that promotes apoptosis. The composition also may be formulated to evaporate slowly from the skin, allowing the agonist extra time to penetrate the skin.

A cream-based topical composition containing a HSP inhibitor or a compound that promotes apoptosis is prepared by mixing together water and all water soluble components in a mixing vessel—A. The pH is adjusted in a desired range from about 3.5 to 8.0. In order to achieve complete dissolution of ingredients the vessel temperature may be raised to up to 45° C. The selection of pH and temperature will depend on the stability of the HSP inhibitor. The oil soluble components, except for the preservative and fragrance components, are mixed together in another container (B) and heated to up to 70° C. to melt and mix the components. The heated contents of vessel B are poured into the water phase (container A) with brisk stirring. Mixing is continued for about 20 minutes. The preservative components are added at temperature of about 40° C. Stirring is continued until the temperature reaches about 25° C. to yield a soft cream with a viscosity of 8,000-12,000 cps, or a desired viscosity. The fragrance components are added at about 25° C.-30° C. while the contents are still being mixed and the viscosity has not yet built up to the desired range. If it is desired to increase the viscosity of the resulting emulsion, shear can be applied using a conventional homogenizer, for example a Silverson L4R homogenizer with a square hole high sheer screen. The topical composition can be fabricated by including the active agent in the water phase during the aforedescribed formulation preparation or can be added after the formulation (vehicle) preparation has been completed. The active agent can also be added during any step of the vehicle preparation. The components of the cream formulations are described in the examples below.

### EXAMPLE #1 (Cream)

| INCI Name | W/w (%) |
|---|---|
| DI Water | 61.00-75.00 |
| Active ingredient^{a} | 1.00-15.00 |
| Mineral oil | 1.90 |
| Glyceryl stearate | 3.60 |
| PEG 100 Stearate | 3.48 |
| Cetearyl Alcohol | 2.59 |
| Ceteareth-20 | 2.13 |
| Dimethicone, 100 ct | 0.48 |
| Lipidure PMB^{b} | 3.00 |
| Advanced Moisture Complex^{c} | 5.00 |
| Stearyl alcohol | 1.42 |
| Preservative, fragrance and color pigment | qs |
| Total | 100.00 |

^{a}An HSP inhibitor or compound that promotes apoptosis.
^{b}Polyquartinium-51 (Collaborative Labs, NY).
^{c}Glycerin, water, sodium PCA, urea, trehalose, polyqauternium-51, and sodium hyaluronate (Collaborative Labs, NY).

### EXAMPLE #2 (Cream)

| INCI Name | w/w (%) |
|---|---|
| Active ingredient^{a} | 0.5-15.00 |
| Glycerol (Glycerin) | 0-5 |
| Isoceteth-20 | 3-7 |
| Glyceryl isostearate | 1.5-5 |
| Dicaprylyl ether | 3-15 |
| Glyceryl triacetate (triacetin) | 0.5-10 |
| Preservative, fragrance and color pigment | q.s. |
| Water | q.s. to 100.00 |

^{a}An HSP inhibitor or compound that promotes apoptosis.

### EXAMPLE #3 (Cream)

| INCI Name | w/w (%) |
|---|---|
| Active ingredient^{a} | 0.5-15.00 |
| Glycerol (Glycerin) | 0-5 |
| Isoceteth-20 | 3-7 |
| Glyceryl isostearate | 1.5-5 |
| Dicaprylyl ether | 3-15 |
| 1-dodecyl-2-pyrrolidanone | 0.5-10% |
| Preservative, fragrance and color | q.s. |
| Water | to 100.00 |

^{a}An HSP inhibitor or compound that promotes apoptosis.

### EXAMPLE #4 (Cream)

| INCI Name | w/w (%) |
|---|---|
| Water | 70 |
| Glyceryl stearate | 4 |
| PEG-100 | 4 |
| Cetearyl alcohol | 3 |
| Ceteareth-20 | 2.5 |
| Mineral oil | 2 |
| Stearyl alcohol | 2 |
| Dimethicone | 0.5 |
| Preservatives | 0.43 |
| 1-Dodecyl-2-pyrrolidanone | 1-10 |
| Total | 100.00 |

An HSP inhibitor or compound that promotes apoptosis is added to the example 4 formulation and mixed until solubilized.

### EXAMPLE #5 (Cream)

| INCI Name | w/w (%) |
|---|---|
| Water | 70-80 |
| Glyceryl Stearate | 4 |
| PEG-100 | 4 |
| Cetearyl alcohol | 3 |
| Ceteareth-20 | 2.5 |
| Mineral oil | 2 |
| Stearyl alcohol | 2 |
| Dimethicone | 0.5 |
| Preservatives | 0.43 |
| Monocaprylate/caprate (Estol | 1-10 |
| 3601, Uniquema, NJ) | |
| Total | 100.00 |

An HSP inhibitor or compound that promotes apoptosis is added to the example 5 formulation and mixed until solubilized.

### EXAMPLE #6 (Cream)

| INCI Name | w/w (%) |
|---|---|
| Water | 70-80 |
| Glyceryl stearate | 4 |
| PEG-100 | 4 |
| Cetearyl alcohol | 3 |
| Ceteareth-20 | 2.5 |
| Mineral oil | 2 |
| Stearyl alcohol | 2 |
| Dimethicone | 0.5 |
| Preservatives | 0.43 |
| cis Fatty acids | 1-10 |
| Total | 100.00 |

An HSP inhibitor or compound that promotes apoptosis is added to the example 6 formulation and mixed until solubilized.

### EXAMPLE #7 (Cream)

| INCI Name | w/w (%) |
|---|---|
| Water | 70-80% |
| Glyceryl stearate | 4 |
| PEG-100 | 4 |
| Cetearyl alcohol | 3 |
| Ceteareth-20 | 2.5 |
| Mineral oil | 2 |
| Stearyl alcohol | 2 |
| Dimethicone | 0.5 |
| Preservatives | 0.43 |
| Terpene(s) | 1-10 |
| Total | 100.00 |

An HSP inhibitor or compound that promotes apoptosis is added to the example 7 formulation and mixed until solubilized.

### EXAMPLE #8 (Cream)

| INCI Name | w/w (%) |
|---|---|
| Water | 70-80% |
| Glyceryl stearate | 4 |
| PEG-100 | 4 |
| Cetearyl alcohol | 3 |
| Ceteareth-20 | 2.5 |
| Mineral oil | 2 |
| Stearyl alcohol | 2 |
| Dimethicone | 0.5 |
| Preservatives | 0.43 |
| Polyoxyethylene sorbitans (tween) | 1-10 |
| Total | 100.00 |

An HSP inhibitor or compound that promotes apoptosis is added to the example 8 formulation and mixed until solubilized.

A hydroalcoholic formulation containing an HSP inhibitor or compound that promotes apoptosis is prepared by mixing the formulation components in a mixing vessel. The pH of the formulation is adjusted to a desired value in the range of 3.5-8.0. The pH adjustment can also be made to cause complete dissolution of the formulation ingredients. In addition, heating can be applied to up to 45° C., or even up to 70° C. depending on the stability of the active in order to achieve dissolution of the formulation ingredients. Several hydroalcoholic formulations are listed below.

### EXAMPLE #9 (Hydro-alcoholic)

| INCI Name | w/w (%) |
|---|---|
| Water | 48.00-62.50 |
| Active ingredient^{a} | 0.5-15.00 |
| Ethanol | 16.00 |
| Propylene glycol | 5.00 |
| Dipropylene glycol | 5.00 |
| Benzyl alcohol | 400 |
| Propylene carbonate | 2.00 |
| Captex-300^{b} | 5.00 |
| Total | 100.00 |

^{a}An HSP inhibitor or compound that promotes apoptosis.
^{b}Caprylic/capric triglyceride (Abitec Corp., OH).

### EXAMPLE #10 (Hydro-alcoholic)

| INCI Name | w/w (%) |
|---|---|
| Water | 53.00-67.9 |
| Active ingredient^{a} | 0.1-15.00 |
| Ethanol | 16.00 |
| Propylene glycol | 5.00 |
| Dipropylene glycol dimethyl ether | 5.00 |
| Benzyl alcohol | 4.00 |
| Propylene carbonate | 2.00 |
| Total | 100.00 |

^{a}An HSP inhibitor or compound that promotes apoptosis.

### EXAMPLE #11 (Hydro-alcoholic)

| INCI Name | w/w (%) |
|---|---|
| Ethanol (alcohol) | 80 |
| Water | 17.5 |
| Propylene glycol dipelargonate | 2.0 |
| Propylene glycol | 0.5 |
| Total | 100.00 |

An HSP inhibitor or compound that promotes apoptosis is added to the formulation and mixed until solubilized.

Heating can be performed, for example, using a laser, flashlamp or an Intense Pulse Light (IPL) device. For example, the device can be a Diode laser in the wavelength range of 700-1300 nm (e.g., 810 nm), a Ruby laser at 654 nm, an Alexandrite laser at 755 nm, a Nd:YAG laser at 1064 nm, a Nd:YAG laser in the range of 600-850 nm, a Pulsed Light, Intense Pulsed Light, or a Flash Lamp in the wavelength range of 400-1200 nm, a Fluorescent Pulsed Light at 550, 580, or 615-1200 nm, a Light Emitting Diode (LED) in the wavelength range of 400-700 nm, and an optical (580-980 nm) or Diode (800±25 nm) energy combined with Radio-Frequency electrical energy. The energy output (J/cm2) of the light and photothermal devices can be, for example, from 0.5-50 J/cm2, 2-20 J/cm2, or 1-10 J/cm2. Other laser and light source parameters that effect heating include pulse duration, spot size and repetition rate. The ranges for these parameters depend on the heating device used. The pulse duration can range, for example, from 0.1 ms to up to 500 ms or it can be a continuous wave (CW) as described in U.S. Pat. No. 6,273,884.

During heating, the temperature of the skin generally is heated to at least 40° C., for example, between 40° C. and 55° C. However, the skin can be heated, as high as, for example, 70° C. using short millisecond pulses, and avoiding skin burn. In addition the temperature at hair follicle or dermal or subdermal skin may reach between 40-150° C. For the lower temperature range of 40-60° C. one should keep the exposure time to between 0.5 min to several minutes. For temperatures above 60° C. one should stay within exposure time of 1-500 msec. The temperature of the skin obtained by heating by a particular mechanism generally can be determined as follows. A subject having a normal body temperature is placed in a room having a temperature of 25° C. A 0.009-inch-diameter thermocouple is placed in an area in the skin. The thermocouple output is connected to a National Instruments SCXI-1112 thermocouple signal conditioner. A National Instruments 6052E data acquisition board, having a maximum acquisition rate of 333 kilo-samples per second controlled the data acquisition and signal gain. The SCXI-1112 and NI 6052E DAQ combination could simultaneously detect up to eight thermocouple outputs at a rate of 42 kilo-samples per second. The sampling rate can be conducted, for example, at 1000 samples/sec.

A similar method is used to determine the temperature range in the hypodermal region. In this case, a thermocouple is inserted to a depth of about 5 mm in an ex-vivo human skin and treatment is applied at the skin surface.

The composition should be applied topically to a selected area of skin from which it is desired to reduce hair growth within 14 days (before of after) of heating. The time period for the topical composition application, before or after, the heating treatment may vary from as short as 1 day or even a simultaneous application, depending on the nature of the active chemical in the topical composition. Preferably, the composition is applied (multiple, for example, two, three, or four times) within seven days of heating, and more preferably at least once within one or two days of heating. Compositions can be applied once a day for at least two or three days prior to heating.

The composition, for example, can be applied to the face, particularly to the beard area of the face, i.e., the cheek, neck, upper lip, and chin. The composition/heating combination may be used as an adjunct to other methods of hair removal including shaving, waxing, mechanical epilation, chemical depilation, and electrolysis. The composition can also be applied to the legs, arms, torso or armpits. The composition is particularly suitable for reducing the growth of unwanted hair in women having hirsutism or other conditions.

Reduced hair growth can be demonstrated quantitatively by reduced hair length, hair diameter, hair pigmentation, and/or hair density in the treated area. Reduced hair growth can be demonstrated cosmetically by less visible hair, shorter hair stubble, finer/thinner hair, softer hair, and/or a longer-lasting shave in the treated area.

Golden Syrian Hamster Assay

Male intact Golden Syrian hamsters are considered acceptable models for human beard hair growth in that they display oval shaped flank organs, one on each side, each about 8 mm. in major diameter. These organs produce fine light colored hair typical of the animal pelage found on the body. In response to androgens the flank organs produce dark coarse hair similar to male human beard hair. To evaluate the effectiveness of a composition in reducing hair growth (including when used in conjunction with heating), the flank organs of each of a group of hamsters are depilated by applying a thioglycolate based chemical depilatory (Surgex) and/or shaved. To one organ of each animal 10 μl. of vehicle alone once a day is applied, while to the other organ of each animal an equal amount of vehicle containing the HSP inhibitor or compound that promotes apoptosis under evaluation is applied. After three weeks of topical applications (one application per day for five days a week), with heating at a selected time(s) if the composition is used in conjunction with heating) the flank organs are shaved and the amount of recovered hair (hair mass) from each is weighed. Percent-reduction of hair growth is calculated by subtracting the hair mass (mg) value of the test compound treated side from the hair mass value of the vehicle treated side; the delta value obtained is then divided by the hair mass value of the vehicle treated side, and the resultant number is multiplied by 100.

Human Hair Follicle Growth Assay:

Human hair follicles in growth phase (anagen) were isolated from face-lift tissue (obtained from plastic surgeons) under dissecting scope using a scalpel and watchmakers forceps. The skin was sliced into thin strips exposing 2-3 rows of follicles that could readily be dissected. Follicles were placed into 0.5 ml William's E medium (Life Technologies, Gaithersburg, Md.) supplemented with 2 mM L-glutamine, 10 μg/ml insulin, 10 ng/ml hydrocortisone, 100 units of penicillin, 0.1 mg/ml streptomycin and 0.25 μg/ml amphotericin B. The follicles were incubated in 24-well plates (1 follicle/well) at 37° C. in an atmosphere of 5% CO₂ and 95% air. The HSP inhibitor or compound that promotes apoptosis is dissolved into dimethyl sulfoxide as 100-fold stock solution. The control hair follicles were treated with dimethyl sulfoxide without the inhibitor/compound. The follicles were photographed in the 24-well plates under the dissecting scope at a power of 10×. The follicles also are heated at a selected time(s) if the composition is used in conjunction with heating. Typically, image recordings were made on day 0 (day follicles were placed in culture), and again on day 7. The length of hair follicle was assessed using an image analysis software system. The growth of hair fiber was calculated by the subtracting the follicle length on day 0 from that determined on day 7.

### EXAMPLE 1

KNK437 was tested in the human hair follicle growth assay. Four groups of hair follicles were used in the experiment. The first group of hair follicles did not receive any treatments (control). In the second group, the hair follicles were treated with KNK 437 (5-20 μM). In the third group, hair follicles were irradiated with a laser (0.75W-0.82W/100 msec) on day 2 of the experiment. In the last group, the hair follicles were treated for 24 hours with KNK 437 (5-20 μM) prior to the laser treatment (0.75W-0.82W/100 msec), with continued treatment of 5 μM KNK 437. Hair growth was measured after seven days.

The combined treatment (fourth group) provided between a 46% and 66% reduction in hair growth. The laser by itself, using the same conditions, provided between a 2% and 22% reduction in hair growth. Treatment with only KNK 437 provided between a 27% and 55% reduction in hair growth.

### EXAMPLE 2

Caffeine also was tested in the human hair follicle growth assay. Hair follicles were pre-incubated for 24 hours with 0.1 mM concentration of caffeine, and then some of the follicles were laser treated at 0.75W for 100 msec using a Coherent diode laser system. The length of each follicle was measured and the follicles then were placed in a 37° C. incubator for four to six days. Following incubation the hair shaft growth was measured. Only the follicles treated with the caffeine solution in combination with the laser exhibited significant reduction in hair growth compared with the control group.

Other embodiments are within the claims. For example, the classes of compounds and specific compounds described in the list of patents above and incorporated by reference can be used in the methods disclosed in the Summary section. Thus, for example, Example 3 provides the reduction in hair growth achieved using the combination of a lipoxygenase inhibitor and a laser.

### EXAMPLE 3

A composition including a vehicle 15% DMSO, 65% ethanol, 10% propylene glycol, and 10% di-propylene glycol and 5% quercetin by weight was tested in the Golden Syrian Hamster assay. Animals were treated with either 5% quercetin compared to vehicle control site (without quercetin), 15W/0.75 sec laser compared to the untreated control site, or 5% quercetin for four days prior to 15W/0.75 sec laser versus 15W/0.75 sec laser alone.

Three weeks later the hamster flank organs treated only with 5% quercetin, 15% hair growth inhibition was observed compared to the vehicle control flank organs. Flank organs irradiated with 15W/0.75 sec laser alone no hair growth inhibition compared to the untreated control site. In hamster flank organs, which were pretreated with 5% quercetin for four days prior to 15W/0.75 sec laser, hair growth inhibition was 36% compared to flank organs, which were irradiated with the same laser pulse without quercetin pretreatment.

1. A method of reducing hair growth in a mammal, comprising:
(a) selecting an area of skin on the mammal from which reduced hair growth is desired;
(b) applying to the area of skin a dermatologically acceptable composition comprising a heat shock protein inhibitor; and
(c) heating the area of skin within 14 days of step (b).

2. The method of claim 1, wherein a laser is used for step (c).

3. The method of claim 1, wherein a flashlamp is used for step (c).

4. The method of claim 1, wherein the composition includes between 0.1% and 30% of the inhibitor by weight.

5. The method of claim 1, wherein the area of skin is on the face, axilla, torso, and/or leg of a human.

6. The method of claim 1, wherein the inhibitor can inhibit the activation of one or more hair follicle heat shock proteins.

7. The method of claim 1, wherein the inhibitor can bind to one or more hair follicle heat shock proteins.

8. The method of claim 1, wherein the inhibitor can reduce the level and/or expression of one or more heat shock proteins in hair follicles.

9. The method of claim 1, wherein the inhibitor can reduce the expression of one or more heat shock proteins mRNA's in hair follicles.

10. The method of claim 1, wherein the inhibitor is KNK 437

11. The method of claim 1, wherein the inhibitor is geldanomycin.

12. A method of reducing hair growth in a mammal, comprising:
(a) selecting an area of skin on the mammal from which reduced hair growth is desired;
(b) applying to the area of skin a dermatologically acceptable composition comprising an a compound that promotes apoptosis; and
(c) heating the area of skin within 14 days of step (b).

13. The method of claim 12, wherein a laser is used for step (c).

14. The method of claim 12, wherein the composition includes between 0.1% and 30% of the compound by weight.

15. The method of claim 12, wherein the area of skin is on the face of a human.

16. The method of claim 12, wherein the compound is a methylxanithine.

17. The method of claim 12, wherein the compound is caffeine.

18. A method of reducing hair growth in a mammal, comprising:
(a) selecting an area of skin on the mammal from which reduced hair growth is desired;
(b) applying to the area of skin a dermatologically acceptable composition comprising an inhibitor of a heat shock protein; and
(c) treating the area skin with a laser, a flashlamp, or an IPL device within 14 days of step (b).

19. A method of reducing hair growth in a mammal, comprising:
(a) selecting an area of skin on the mammal from which reduced hair growth is desired;
(b) applying to the area of skin a dermatologically acceptable composition comprising an a compound that promotes apoptosis; and
(c) treating the area of skin with a laser, a flashlamp, or an IPL device within 14 days of step (b).
